(19) United States
(12) Patent Application Publication
(10) Pub. No.: US 2023/0101766 A1
(43) Pub. Date: Mar. 30, 2023

(54) TREATMENT METHOD FOR LUMINAL ORGAN

(71) Applicant: OLYMPUS MEDICAL SYSTEMS CORP., Tokyo (JP)

(72) Inventors: Tomofumi KATAYAMA, Kunitachi-shi (JP); Rei MATSUNAGA, Hino-shi (JP); Yuji KISHIMOTO, Kodaira-shi (JP); Kengo SASAKI, Kokubunji-shi (JP)

(73) Assignee: OLYMPUS MEDICAL SYSTEMS CORP., Tokyo (JP)

(21) Appl. No.: 17/953,658

(22) Filed: Sep. 27, 2022

Related U.S. Application Data
(60) Provisional application No. 63/249,208, filed on Sep. 28, 2021.

(51) Int. Cl. A61N 5/06; A61N 5/067

(52) U.S. Cl. CPC A61N 5/0603; A61N 5/067

(57) ABSTRACT

A method of cholecystitis treatment according to an aspect of the present disclosure comprises a step A including contacting inner walls of a gallbladder to each other by constricting the gallbladder and a step B including maintaining the inner walls of the gallbladder contacting each other.

### RELATED APPLICATION DATA

This application is based on and claims priority under 37 U.S.C. § 119 to U.S. Provisional Application No. 63/249,208 filed on Sep. 28, 2021, the entire contents of which are incorporated herein by reference.

### FIELD OF THE DISCLOSURE

The present disclosure relates to a treatment method for a luminal organ and, in more detail, a treatment method for a sac-shaped portion in a luminal organ.

### BACKGROUND

In the bile duct system, which is a luminal organ, the gallbladder is present as a sac-shaped portion.

Acute cholecystitis, which is one of the diseases of the gallbladder, often occurs in a case where calculi formed in the gallbladder move to the cystic duct.

The current standard treatment of acute cholecystitis is laparoscopic cholecystectomy. In laparoscopic cholecystectomy, there is no risk of recurrence because the gallbladder is removed.

In the treatment of cholecystitis, a treatment method less invasive than laparoscopic cholecystectomy has been sought. U.S. Pat. No. 8,460,314 discloses a method of puncturing the gallbladder from the gastrointestinal tract under an ultrasonic endoscope and eliminating the function of the inner mucosa membrane of the gallbladder by cauterization or the like. In a case where the function of the inner mucosa membrane of the gallbladder is eliminated, bile is not concentrated, and thus the generation and growth of calculi are suppressed.

### BRIEF SUMMARY OF EMBODIMENTS

A first aspect of the present disclosure is a method of cholecystitis treatment.

This treatment method comprises a step A including contacting inner walls of a gallbladder to each other by constricting the gallbladder and a step B including maintaining the inner walls of the gallbladder contacting each other.

A second aspect of the present disclosure is a treatment method for an organ.

This treatment method comprises a step A including contacting inner walls of an organ to each other and a step B including maintaining the inner walls of the organ contacting each other.

### BRIEF DESCRIPTION OF THE DRAWINGS

FIG. 1 is a view showing a portion of a gallbladder.

FIG. 2 is a view showing an example of an access operation to the gallbladder.

FIG. 3 is a view showing an example of an access operation to the gallbladder.

FIG. 4 is a view showing a state in which the gallbladder are communicated with the duodenum through a stent.

FIG. 5 is a view showing an example of a step A according to an embodiment of the present disclosure.

FIG. 6 is a view showing an example of a step B according to an embodiment of the present disclosure.

FIG. 7 is a view showing an example of a change in the gallbladder in a route A.

FIG. 8 is a view showing an example of a change in the gallbladder in a route B.

FIG. 9 is a view showing an example of a change in the gallbladder in a route C.

### DETAILED DESCRIPTION

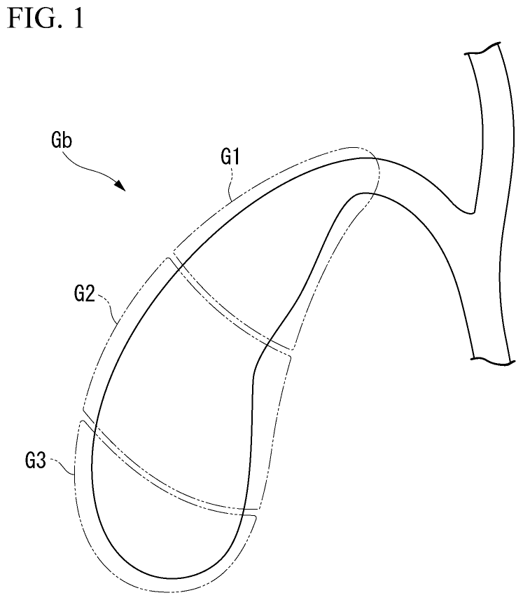

An embodiment of the present disclosure will be described with reference to FIG. 1 to FIG. 9. This embodiment is an example of treating cholecystitis.

First, the contents of the gallbladder are removed. The contents of the gallbladder are mainly bile and gallstone (calculi). Bile is removed by gallbladder drainage, and calculi are removed using various treatment tools.

There are roughly three kinds of access routes to the gallbladder for performing gallbladder drainage, and any route may be used in the embodiment.

In the transcutaneous access route (hereinafter, may be referred to as a “route A”), the abdominal wall is punctured while confirming the position of the gallbladder with an X-ray fluoroscopic image, and a puncture needle is inserted into the gallbladder via the liver parenchyma. Then, a drainage tube is indwelled by a known procedure using a guide wire or the like, and a drainage route is established. The distal end of the drainage tube may be located in the gallbladder or may be located in the liver parenchyma.

After drainage, the drainage tube is removed with the guide wire being indwelled, and a balloon catheter is inserted. In order for the cholangioscope Cs to be capable of being inserted, which will be described later, the fistula on the access route is extended with a balloon catheter to the same size as the diameter of the cholangioscope Cs. An over-tube or the like is indwelled in the extended fistula to make the access route reliable. Then, the cholangioscope Cs is inserted into the over-tube.

FIG. 1 shows the portion of the gallbladder Gb. In the route A, a drainage route may be established in any one of a cervical part (an opening) G1, a body part G2, and a bottom part G3 of the gallbladder Gb.

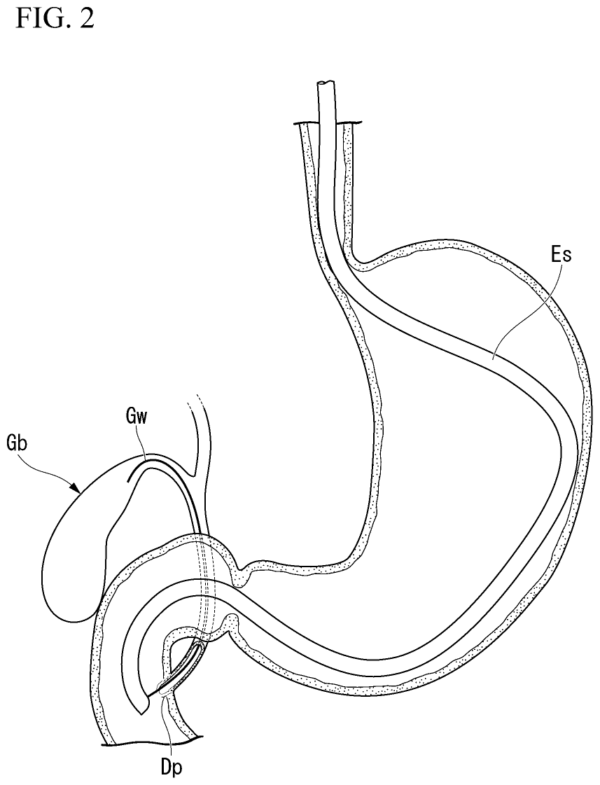

In the transpapillary access route (hereinafter, may be referred to as a “route B”), as shown in FIG. 2, a guide wire Gw is inserted into the duodenum in the same procedure as the ERCP (endoscopic retrograde cholangiopancreatography) from an endoscope Es introduced into the duodenal papilla Dp. Then, performing that the distal end of the guide wire Gw is made to reach the inside of the gallbladder Gb, and a drainage tube is indwelled along the guide wire.

In the transabdominal access route (hereinafter, may be referred to as a “route C”), performing that a puncture needle protruded from the endoscope is inserted into the duodenal wall while confirming the position of the gallbladder with an image of the ultrasonic endoscope introduced into the duodenum and further inserted into the gallbladder. Then, in a state in which the puncture needle is inserted into the gallbladder, performing that the guide wire is passed through the puncture needle, the distal end of the guide wire is made to reach the inside of the gallbladder, and then only the puncture needle is removed with the guide wire being left. Then, a drainage tube is indwelled along the indwelled guide wire.

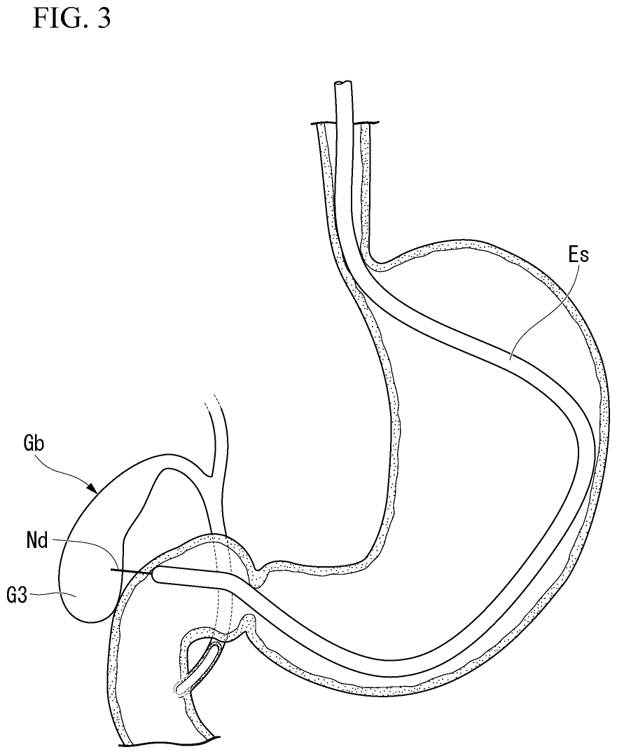

In the route C, a drainage route may be established in any one of the cervical part G1, body part G2, and bottom part G3 of the gallbladder Db. However, it is preferable that a drainage route is established in the bottom part G3 in consideration of the subsequent procedure. In a case where a drainage route is established at the bottom part G3, as shown in FIG. 3, performing that a puncture needle Nd protruded from the endoscope Es is inserted into the bottom part G3 of the gallbladder Gb. In a state in which the puncture needle is inserted into the gallbladder, performing that the guide wire is passed through the puncture needle Nd, the distal end of the guide wire is reached the inside of the gallbladder, and then only the puncture needle is removed with the guide wire being left.

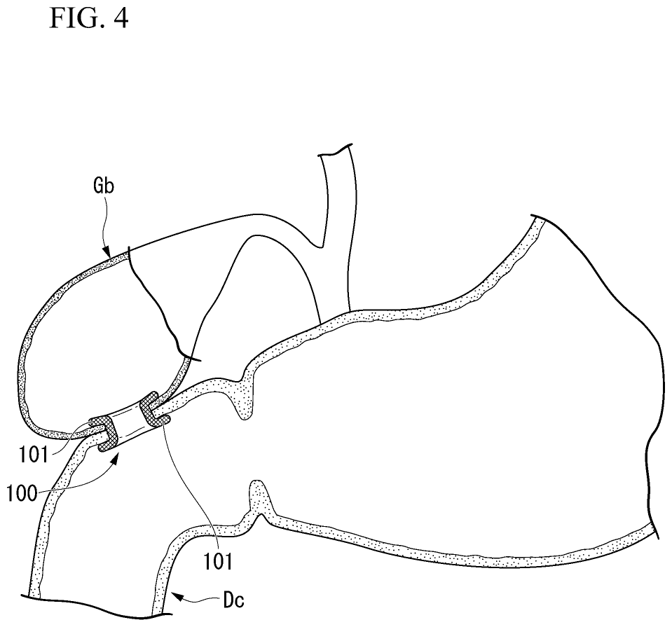

Then, it is preferable to make the distal end of the stent delivery device to reach the gallbladder along the indwelled guide wire and a covered stent connecting the gallbladder and the duodenum is indwelled as shown in FIG. 4 in consideration of the possibility that the gallbladder and the duodenum become apart from each other. A covered stent 100 shown in FIG. 4 has flanges 101 on both sides in the axial direction of the tubular shape, and it is configured to be difficult to come out of the gallbladder Gb and the duodenum Dc after indwelling. A covered stent without a flange 101 can also be used. However, in that case, it is preferable to suture the covered stent to the gallbladder or duodenum so that the covered stent is not out of place.

In a case where there are calculi in the gallbladder after the drainage is complete, the calculi are removed as necessary.

The calculi can be removed by various known methods. Specific examples thereof include removal and crushing of calculi with a basket, removal of calculi by suction with a suction catheter, and crushing of calculi with a laser of a laser irradiation device. The calculi may be removed outside the gallbladder in the original form of the calculi or may be removed by making it smaller than the original form by crushing or the like.

After the removal of the contents is completed, the inside of the gallbladder may be washed with a physiological saline solution or the like.

Next, the inner mucosa membrane in the gallbladder is treated to reduce the function of the inner mucosa membrane. Any treatment of a physical treatment using a laser irradiation device, a heat probe, or the like, or a chemical treatment in which an alkaline chemical liquid or the like is sprayed using a catheter or the like can be applied.

In the physical treatment, electrical energy thermally damages tissue. In a case of damaging the tissue, it is preferable to perform cauterization after performing setting to a coagulation wave mode which outputs heat energy of an extent required for melting the tissue without completely incising the tissue. In a case of being set to the coagulation wave mode, it is possible to damage the tissue to the extent necessary while preventing postoperative perforation where heat remains and perforates postoperatively.

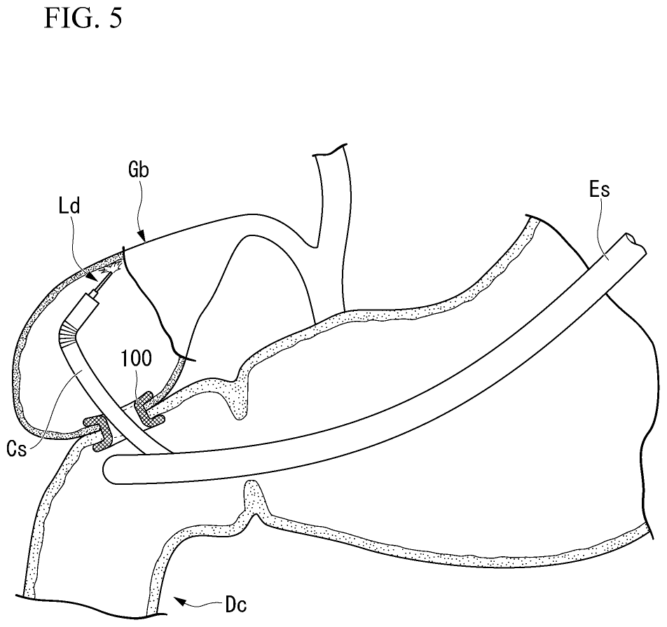

FIG. 5 shows an example of an inner mucosa membrane treatment in the route C. In FIG. 5, performing that a cholangioscope Cs capable of being bendably operated is protruded from the treatment tool channel of the endoscope Es introduced into the duodenum Dc, thereby inserting into the gallbladder Gb via the stent 100. Further, performing that a laser irradiation device Ld is protruded from the treatment tool channel of the cholangioscope Cs to perform a physical treatment. It is also possible to insert the endoscope Es directly into the gallbladder via the stent 100 and cause the laser irradiation device Ld to protrude, thereby performing a treatment without using the cholangioscope Cs.

The cholangioscope Cs capable of being bendably operated can also be used in the route A and the route B.

The chemical treatment can be performed by using a catheter that is protruded from the endoscope Es. However, it is preferable a treatment using a catheter being protruded from the cholangioscope Cs that has entered the gallbladder, because it is possible to damage all around the inner mucosa membrane while observing the inside of the gallbladder.

Examples of the chemical liquid that is used for the chemical treatment include alkyldiaminoethylglycine and alkylpolyaminoethylglycine hydrochloride. In a case where the opening connected to the cystic duct is blocked with a clip or the like before the chemical treatment, it is possible to suppress the damage of the cystic duct due to the chemical liquid. In a case where the gallbladder is subjected to suction to be contracted at the time of the chemical treatment, it is possible to perform the chemical treatment with a smaller amount of chemical liquid.

In a case where the chemical treatment is performed, the washing may be performed after the chemical treatment is completed, without performing the washing after the removal of the contents.

After the treatment of damaging the inner mucosa membrane is completed, mucosal cells are cauterized so that they are in a molten state in a case of the physical treatment, and mucosal cells are in a dead state in a case of the chemical treatment. As a result, the function of the inner mucosa membrane is eliminated or reduced, and the ability to cause calculi to grow is lost or significantly reduced. As a result, in the inside of the gallbladder, new calculi are difficult to be generated, or calculi are difficult to grow.

Next, the gallbladder is contracted and it makes inner walls of the gallbladder to be contacted to each other (a step A). The contraction of the gallbladder can be performed by subjecting the inside of the gallbladder to suction to make it into negative pressure or by hooking a thread on the inner wall of the gallbladder to contractively sew the inner wall. In a case where the cystic duct is blocked with a clip or by cauterization in advance before suction, it is possible to sufficiently perform suction, which is preferable.

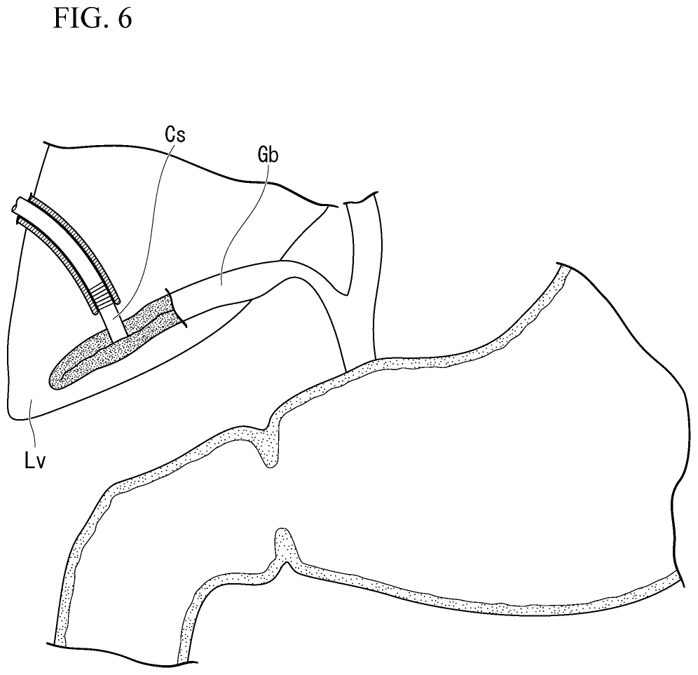

As an example, FIG. 6 shows a state in which the step A is performed by suction using the cholangioscope Cs in the route A via the liver Lv.

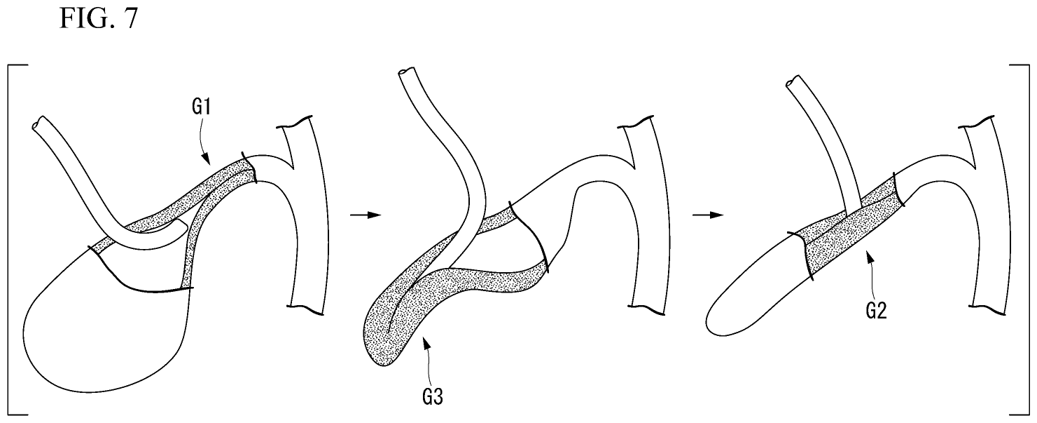

In a case of accessing the bottom part of the gallbladder in the route A, an operator subjects the inside of the gallbladder to suction in the step A to contact the gallbladder in an order of the cervical part G1, the bottom part G3, and the body part G2 as shown in FIG. 7. It is also possible to perform contacting in an order of the bottom part G3, the cervical part G1, and the body part G2.

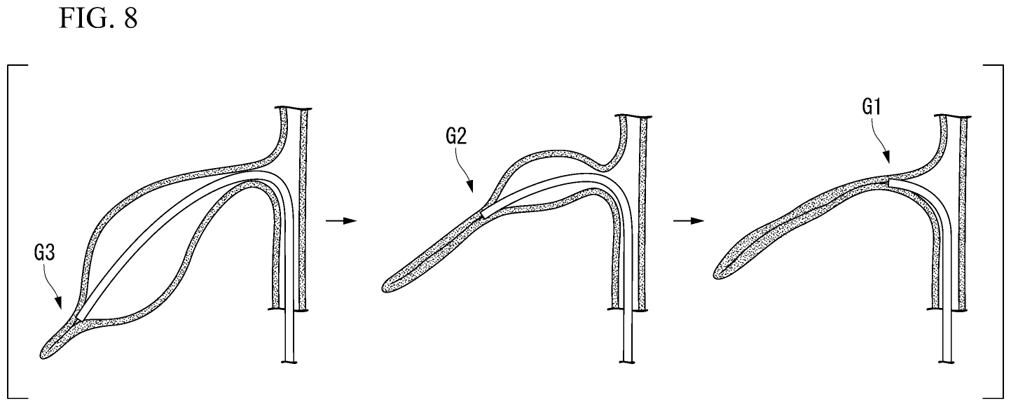

In a case of using the route B, the operator contacts the gallbladder in an order of the bottom part G3, the body part G2, and the cervical part G1 as shown in FIG. 8. In a case of finally contacting the cervical part G1 which is an access route, it is possible to maintain a state in which the field of view is secured until the endoscope Es or the cholangioscope Cs is removed from the gallbladder Gb.

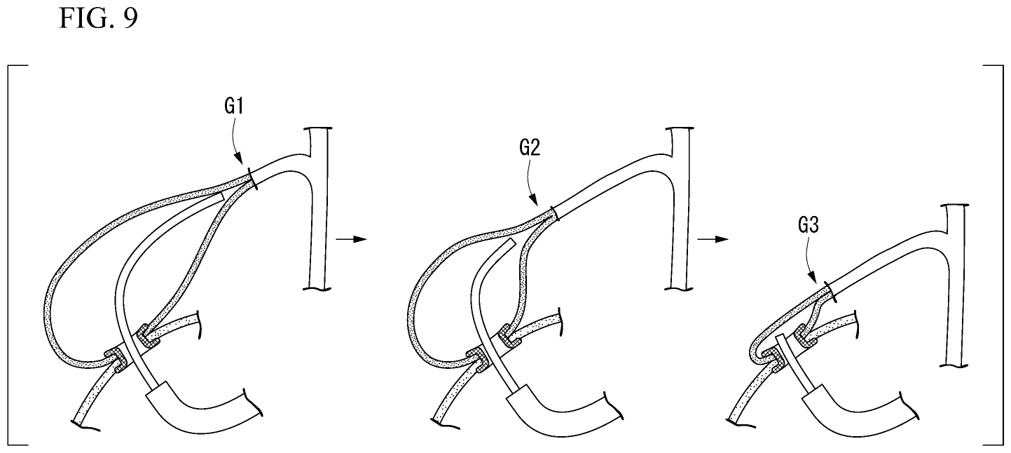

In a case of using the route C, the operator contacts the gallbladder in an order of the cervical part G1, body part G2, and bottom part G3 as shown in FIG. 9. In a case of finally contacting the bottom part G3 which is an access route, it is possible to maintain a state in which the field of view is secured until the endoscope Es or the cholangioscope Cs is removed from the gallbladder Gb.

In a case of contractively sewing the inner wall of the gallbladder in the step A, a device of a needle holder that is protruded from the endoscope Es or the cholangioscope Cs, a needle thread, and the like is used.

After the completion of the step A, it may be confirmed that the gallbladder is contacted by observing the inside of the gallbladder. As the observation means, abdominal echography or the like can be used particularly in the route A, and an ultrasonic endoscope or the like can be used particularly in the routes B and C.

Then, a state in which the inner wall of the gallbladder is contacted is maintained for a predetermined period of time (a step B). The period of time of maintenance can be, for example, about 1 week to 2 months. The contacting state can be maintained by applying a curing agent onto the inner wall before suction, maintaining the tightened state of the contractively sewn thread, maintaining the negative pressure state by continuing suction, and the like.

Examples of the applicable curing agent include a cyanoacrylate-based curing agent, a polyurethane-based curing agent, gelatin-resorcin-formalin (GRF), which is a bioadhesive, and a fibrin glue.

After the step B, the state where the inner walls of the gallbladder are contacted is maintained, and thus the bile produced by the liver does not enter the gallbladder. As a result, although the gallbladder remains in the body, cholecystitis does not recur.

The treatment of cholecystitis to which the treatment method according to the present embodiment is applied is capable of preventing recurrence of cholecystitis without cholecystectomy. Accordingly, the treatment method is less invasive than cholecystectomy.

It is also disclosed in U.S. Pat. No. 8,460,314 that the function of the inner mucosa membrane of the gallbladder is eliminated by cauterization or the like. However, the effect of suppressing the onset of cholecystitis is temporary since the function of the inner mucosa membrane is restored with an elapse of time.

Since the treatment method of the present embodiment includes the steps A and B, the bile that is the material of the calculi does not enter the gallbladder even in a case where the function of the inner mucosa membrane of the gallbladder is restored. As a result, even after the function of the inner mucosa membrane of the gallbladder is restored, it is possible to keep the effect of suppressing the onset of cholecystitis for a long period of time.

The embodiment of the present disclosure has been described above. However, the technical scope of the present disclosure is not limited to the above embodiment, and it is possible to change the combination of constitutional elements, make various changes to each constitutional element, or delete a constitutional element without departing from the gist of the present disclosure. In addition to the changes described above, some additional changes are exemplified; however, other changes are possible. Two or more of these changes may be combined as appropriate, or they may be combined with the above-described changes.

In the treatment method according to the present disclosure, it is not essential to remove the contents of the gallbladder. Even in a case where the steps A and B have been performed in a state in which the contents remain in the gallbladder, new bile is prevented from entering the gallbladder thereafter, and thus a situation in which the enlarged gallstone moves to the cystic duct does not occur. Therefore, the effect of preventing the onset of cholecystitis is exhibited.

Similarly, washing the inside of the gallbladder is not essential in the treatment method according to the present disclosure. However, in a case where the inside of the gallbladder is sufficiently washed after the physical treatment or the chemical treatment, the remaining tissue, bile, and drugs can be sufficiently discharged. Thereby, the following advantages result. It is possible to make the contacting state of the gallbladder more firm, and it is possible to prevent cholecystitis due to the residual bile, by contacting the gallbladder in a clean state.

The target of the treatment method according to the present disclosure is not limited to the above-described cholecystitis. As a result, it may be applied to other gallbladder diseases other than cholecystitis, and it can also be applied to a sac-shaped portion of the gastrointestinal tract such as the appendix. Furthermore, it can be applied to a sac-shaped portion of other luminal organs other than the gastrointestinal tract.

In the above-described embodiment, suction and suturing are shown as an example of the realization means for contacting the inner walls of the gallbladder, in the step A, and suctioning, suturing, and adhering are shown as examples of the realization means for maintaining the state where the inner walls are closely attached, in the step B. Here, it is not essential that the means for realizing the step A and the means for realizing the step B are the same, and the combination thereof can be freely determined.

What is claimed is:

1. A method of cholecystitis treatment, comprising:
a step A including contacting inner walls of a gallbladder to each other by constricting the gallbladder; and
a step B including maintaining the inner walls of the gallbladder contacting to each other.

2. The method of cholecystitis treatment according to claim 1, wherein the step A includes applying a negative pressure to an inner volume of the gallbladder.

3. The method of cholecystitis treatment according to claim 1, wherein the step A includes suturing the gallbladder using a thread.

4. The method of cholecystitis treatment according to claim 1, wherein the step B includes adhering the inner walls to each other.

5. The method of cholecystitis treatment according to claim 1, the method further comprising removing a content from the gallbladder before the step A.

6. The method of cholecystitis treatment according to claim 1, the method further comprising damaging an inner mucosa membrane of the gallbladder before the step A.

7. The method of cholecystitis treatment according to claim 6, wherein damaging the inner mucosa membrane includes spraying a chemical onto the inner mucosa membrane by using a catheter inserted into the gallbladder.

8. The method of cholecystitis treatment according to claim 6, wherein damaging the inner mucosa membrane includes ablating the inner mucosa membrane with a laser of a laser irradiation device inserted into the gallbladder.

9. The method of cholecystitis treatment according to claim 1, wherein contacting inner walls of the gallbladder to each other in step A includes contacting the inner walls sequentially from a cervical of the gallbladder to a bottom of the gallbladder.

10. The method of cholecystitis treatment according to claim 1, wherein contacting inner walls of the gallbladder to each other in step A includes contacting the inner walls sequentially from a bottom of the gallbladder to a cervical of the gallbladder.

11. The method of cholecystitis treatment according to claim 1, wherein contacting inner walls of the gallbladder to each other in step A includes contacting the internal walls of a body of the gallbladder after contacting the inner walls of a cervical of the gallbladder and contacting the inner walls of a bottom of the gallbladder.

12. A treatment method, comprising:
a step A including contacting inner walls of an organ to each other; and
a step B including maintaining the inner walls of the organ contacting to each other.

13. The treatment method according to claim 12, wherein the step A includes applying a negative pressure to an inner volume of the organ.

14. The treatment method according to claim 12, wherein the step A includes suturing the organ using a thread.

15. The treatment method according to claim 12, wherein the step B includes adhering the inner walls to each other.

16. The treatment method according to claim 12, the method further comprising removing a content from the organ.

17. The treatment method according to claim 12, the method further comprising damaging an inner mucosa membrane of the organ before the step A.

18. The treatment method according to claim 17, wherein damaging the inner mucosa membrane includes spraying a chemical onto the inner mucosa membrane by using a catheter inserted into the organ.

19. The treatment method according to claim 17, wherein damaging the inner mucosa membrane includes ablating the inner mucosa membrane with a laser of a laser irradiation device inserted into the sac-like organ.

20. The treatment method according to claim 12, wherein contacting inner walls of the organ to each other in step A includes contacting the inner walls sequentially from an opening of the organ to a bottom of the organ.

21. The treatment method according to claim 12, wherein contacting inner walls of the organ to each other in step A includes contacting the inner walls of the organ sequentially from a bottom of the organ to an opening of the organ.
